(19) United States
(12) Patent Application Publication
(10) Pub. No.: US 2024/0317824 A1
(43) Pub. Date: Sep. 26, 2024

(54) METHOD FOR PRODUCING CARTILAGE COMPONENT MIXTURE INCLUDING PROTEOGLYCAN

(71) Applicant: ICHIMARU PHARCOS CO., LTD., Gifu (JP)

(72) Inventors: Tatsuji TAKAHASHI, Gifu (JP); Toshiaki FUJINE, Gifu (JP); Lisa WISE, Gifu (JP); Tomoya OKAMOTO, Gifu (JP)

(21) Appl. No.: 18/577,697

(22) PCT Filed: Jun. 28, 2022
(86) PCT No.: PCT/JP2022/025701

§ 371 Date: Jan. 9, 2024

(30) Foreign Application Priority Data
Aug. 6, 2021 (JP) 2021-129997

(51) Int. Cl. C07K 14/46; C07K 1/36; C07K 14/78

(52) U.S. Cl. CPC C07K 14/461; C07K 1/36; C07K 14/78

(57) ABSTRACT

A method for producing a mixture of cartilage components containing proteoglycan, the method including: pulverizing freeze-dried cartilage to obtain a pulverized product; washing the pulverized product with an ethanol solution containing citric acid; washing the product washed with the ethanol solution containing the citric acid further with ethanol; drying and pulverizing the product washed with the ethanol to obtain powder; and wet-pulverizing the obtained powder in water. By using the method, collagens and other cartilage components are extracted efficiently and simultaneously with extraction of proteoglycan from cartilage.

### CROSS-REFERENCE TO RELATED APPLICATIONS

This application claims priority to Japanese Patent Application No. 2021-129997 filed on Aug. 6, 2021 in Japan, the entire contents of which are incorporated herein by reference.

### TECHNICAL FIELD

The present invention relates to a method for producing a mixture of cartilage components containing proteoglycan.

### BACKGROUND ART

Proteoglycan (may also be referred to as “PG”) is a glycoprotein that consists of a single core protein with several to several tens of covalently bonded glycosaminoglycans such as chondroitin sulfate and keratan sulfate, and is widely distributed as one of extracellular matrices in the body such as the skin and cartilage. PG in the cartilage forms an aggregate together with collagen and hyaluronic acid, and a typical cartilage PG is called aggrecan. Aggrecan has a core protein with a large number of glycosaminoglycan sugar chains bonded thereto, and hyaluronic acid and a link protein are bound at its N-terminus.

Glycosaminoglycan has a long linear structure without branches, is negatively charged by having a large number of sulfate groups and carboxyl groups, and has an elongated shape due to its electric repulsion. PG retains a large amount of water due to sugar having an affinity for water and bears functions specific to the cartilage such as elasticity and resistance to impact. Further, PG has been found to have many physiological functions such as an anti-inflammatory action, an action of accelerating hyaluronic acid synthesis, and an epidermal growth factor (EGF)-like action, and is expected to be applied to foods and cosmetics.

Methods for efficiently producing high purity PG have been studied so far. For example, a method of extracting PG from salmon nasal cartilage using acetic acid (see Patent Document 1) and a method of extracting PG by controlling an extraction temperature and stirring speed of an acetic acid solution (see Patent Document 2) have been known. In addition, a method of extracting PG using an aqueous citric acid solution (see Patent Document 3), a method of efficiently producing high purity proteoglycan from a cartilage tissue by extraction in the presence of a specific acidic solution or a protease at a predetermined concentration (see Patent Document 4) have also been reported. The cartilage also contains other cartilage components than proteoglycan such as collagen and hyaluronic acid, and these components are expected to be simultaneously extracted and used as materials for functional foods and cosmetics.

### CITATION LIST

### Patent Documents

Patent Document 1: Japanese Patent No. 3731150
Patent Document 2: Japanese Patent No. 6317053
Patent Document 3: Japanese Patent No. 5749067
Patent Document 4: Japanese Unexamined Patent Publication No. 2020-127397

### SUMMARY OF THE INVENTION

### Technical Problem

An object of the present invention is to efficiently extract other cartilage components such as collagen simultaneously with the extraction of PG from cartilage.

### Solution to the Problem

As a result of intensive studies to achieve the object, the prevent inventors have found a method for producing a mixture of cartilage components containing proteoglycan by optimizing or combining various conditions such as the concentration of an extraction solution and an extraction method. Specifically, the present invention includes the following embodiments.
(1) A method for producing a mixture of cartilage components containing proteoglycan, the method including: pulverizing freeze-dried cartilage to obtain a pulverized product: washing the pulverized product with an ethanol solution containing citric acid (including degreasing, purification, and other processes); washing the product washed with the ethanol solution containing the citric acid further with ethanol: drying and pulverizing the product washed with the ethanol to obtain powder; and wet-pulverizing the obtained powder in water. In a preferred embodiment, the method further includes, after the wet-pulverizing of the powder in water, removing foreign substances from the wet-pulverized aqueous solution.
(2) The method of (1), wherein the ethanol solution containing the citric acid contains the citric acid from greater than or equal to 1% by mass to less than or equal to 30% by mass (1% to 30% by mass).
(3) The method of (1) or (2), wherein the mixture of the cartilage components contains the proteoglycan of 30% by mass or more on a solids basis.
(4) The method of any one of (1) to (3), wherein the mixture of the cartilage components contains water-soluble collagens of at least 20% by mass on a solids basis.
(5) The method of any one of (1) to (4), wherein a molecular weight of the proteoglycan is 800 k to 900 k.
(6) A method for producing a mixture of cartilage components containing proteoglycan, the method including: immersing cartilage in an aqueous acetic acid solution with a concentration of acetic acid in water of 0.03% or more to less than 4% by mass to obtain a cartilage component extract; and collecting the cartilage components from the extract.
(7) The method of (6), wherein the concentration of the acetic acid in water of the aqueous acetic acid solution is 0.5% to 3% by mass.
(8) The method of (6) or (7), wherein the mixture of the cartilage components contains collagens, a content of the collagens being at least 20% by mass on a solids basis.
(9) The method of any one of (6) to (8), wherein a molecular weight of the proteoglycan is 400 k to 650 k.
(10) A method for producing a mixture of cartilage components containing proteoglycan, the method including: immersing cartilage in an aqueous citric acid solution with a concentration of citric acid in water of 0.01% or more to less than 0.05% by mass at 30 degrees C. to 80 degrees C. to obtain a cartilage component extract; and collecting the cartilage components from the extract.
(11) The method of (10), wherein a molecular weight of the proteoglycan is 500 k to 900 k.
(12) The method of any one of (1) to (11), wherein the cartilage is nasal cartilage of a salmon head.
(13) A method for producing a mixture of cartilage components containing proteoglycan, the method including: immersing cartilage in water or an aqueous solution of pH 2.0 to 4.0 to obtain a cartilage component extract; and collecting the cartilage components from the extract.

### Advantages of the Invention

The method of the present invention can efficiently produce a mixture of proteoglycan and other cartilage components such as collagens.

### BRIEF DESCRIPTION OF THE DRAWINGS

FIG. 1 is a flowchart of a production method of a first embodiment.

FIG. 2 is a flowchart of a production method of a second or third embodiment.

### DESCRIPTION OF EMBODIMENTS

Embodiments of the present invention will be described below with reference to the drawings. The embodiments described below do not limit the claimed invention. All the components described in the embodiments and combinations thereof are not necessarily essential to achieve the present invention.

Although the present invention is directed to a production method for obtaining a mixture of cartilage components containing proteoglycan, the mixture of the cartilage components will be described first, and then a method for producing the mixture of the cartilage components will be described in detail.

### (Mixture of Cartilage Components)

The production method of the present invention produces a mixture containing proteoglycan and other cartilage components. Examples of the other cartilage components than proteoglycan include, but are not limited to, collagens and hyaluronic acid. Cartilage is one of connective tissues present in, for example, the nose, ribs, joints, around the trachea, ear conch, and intervertebral discs of vertebrates, and refers to a complex of an extracellular matrix and chondrocytes. The extracellular matrix in the cartilage may also be referred to as a cartilage matrix. The cartilage matrix contains collagens, chondroitin sulfate, hyaluronic acid, and proteoglycan as main components.

Proteoglycan is a complex polysaccharide having a core protein with covalently bonded glycosaminoglycans such as chondroitin sulfate and dermatan sulfate, and is abundantly present in animal tissues, particularly in cartilage tissues. It is also known that proteoglycan exists with its core protein further bound to hyaluronic acid in a living body, and has a molecular weight as high as several tens of thousands to several tens of millions. A typical cartilage-derived proteoglycan is referred to as aggrecan.

Collagens each have a helical structure that consists of three polypeptide molecules each including amino acids linked in a chain form and having a molecular weight of about 100 k, and forms a fibrous or membranous structure. The types and number of amino acids forming collagen are extremely characteristic. As one of the characteristics, collagen contains amino acids such as hydroxyproline and hydroxylysine which are not included in the 20 basic amino acids constituting general proteins. These amino acids are special amino acids contained only in collagen and a limited number of proteins closely related to collagen, and in particular, hydroxyproline accounts for about 10% of the total amino acids in collagen. Thus, hydroxyproline can be considered as a measure of the amount of collagen. Although collagen may be of any type irrespective of the difference in amino acid composition, type II collagen is preferable as collagen contained in a large amount in the cartilage.

Hyaluronic acid is a mucopolysaccharide polymer compound having a chain structure with disaccharide units of N-acetylglucosamine and glucuronic acid bonded and connected together. Examples of other cartilage components include laminin, fibronectin, and elastin.

### First Embodiment

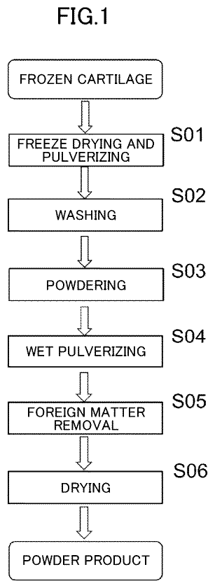

By a method for producing a mixture of cartilage components containing proteoglycan of the first embodiment of the present invention, a component that is hardly soluble in water can be efficiently extracted to obtain a soluble component. FIG. 1 is a flowchart of a typical embodiment of this production method. The method of the first embodiment includes: a freeze-drying and pulverizing step (S01) of freeze-drying and pulverizing frozen cartilage: a washing step (S02) of washing (including degreasing, purification, and other processes) the obtained pulverized product; and a powdering step (S03) of drying and pulverizing the washed pulverized product to obtain powder.

In the present embodiment, the frozen cartilage used as a starting material may be, for example, a cartilage tissue of fishes, mollusks, birds, or mammals, and is preferably a fish cartilage tissue, particularly nasal cartilage of a salmon head. In the present embodiment, for example, cartilage derived from the nasal cartilage tissue contained in the head of fish of Salmonidae family are suitably used in terms of availability and cost. For example, when salmon (mainly Chum salmon) is caught and processed into various products, the head of the salmon to be disposed can be used.

In the freeze-drying and pulverizing step (S01), the cartilage is pulverized after a large amount of water contained in the cartilage is removed. The freeze-drying may be performed by any method, but is preferably performed at a product temperature of 35° C. or lower. The cartilage having its mass reduced to about one tenth by the freeze-drying is pulverized to an appropriate size. The pulverizing is, for example, to pulverize the cartilage to coarse powder having a particle diameter of about 10 mesh to about 16 mesh. The pulverizing may be performed by any method. For example, a comb-type disintegrator, a roller-type pulverizer, a mortar, a jaw crusher, a roller mill, or a jet mill may be used to obtain a pulverized product having an appropriate size.

In the washing step (S02), the obtained pulverized product is dispersed in ethanol to remove lipids and other substances. The volume of ethanol used and time for dispersing the product in ethanol can be appropriately adjusted to remove fat contained in the cartilage as a raw material as much as possible. For example, the pulverized product can be efficiently washed by adding ethanol in an amount of about ten times the volume of the pulverized product used and stirring the mixture at about 50 degrees C. for about 30 minutes to about an hour. The washing with ethanol is preferably performed plural times, and citric acid or a salt thereof is preferably added to an ethanol solution initially used. The addition of citric acid or a salt thereof allows efficient removal of lipids and other substances, advantageously keeping the product from emitting odor due to oxidation of lipids during storage. The lower limit of the amount of citric acid added is not limited to a particular value, but is preferably 1% by mass or more, more preferably 3% by mass or more, yet more preferably 5% by mass. The upper limit of the amount of citric acid added is not also limited to a particular value, but is preferably 30% by mass or less, more preferably 20% by mass or less, yet more preferably 15% by mass or less.

The pulverized product thus washed with ethanol is further dried and pulverized in the powdering step (S03). As a result, the product turns to, for example, finer powder having a particle diameter of about 48 mesh to 80 mesh. The drying for evaporating ethanol may be performed by any method, and the obtained powder may only be left at room temperature. However, the powder is preferably dried under reduced pressure at a heating temperature of 35 degrees C. or lower for more efficient drying. The pulverizing may also be performed by any method, for example, by using an impact mill such as a hammer mill or a pin mill, a medium mill such as a ball mill or a tower mill, or a dry pulverizer such as a jet mill.

The production method of the present embodiment further includes: a wet-pulverizing step (S04) of wet-pulverizing the ethanol-washed powder with water added: a foreign matter removal step (S05) of removing insoluble matter from the wet-pulverized aqueous solution; and a drying step (S06) of drying the obtained aqueous solution.

The wet-pulverizing in the wet-pulverizing step (S04) is to mechanically pulverize the ethanol-washed powder dispersed in water. As an apparatus used for the wet-pulverizing, for example, a stirrer such as a homomixer, a dispersion mixer, an ultramixer, CLEARMIX (trade name) manufactured by M Technique Co., Ltd., and Masscolloider, an ultrasonic homogenizer, and a high-pressure homogenizer can be used. The cartilage component pulverized in this step is soluble in water, and most of it can be collected as an aqueous solution.

The foreign matter removal step (S05) is an optional step preferably included, and is performed to remove foreign matters mixed during the production from the aqueous solution obtained in the wet-pulverizing step.

In the drying step (S06), the aqueous solution that has gone through the step (S05) is dried under predetermined conditions to obtain dry powder. The drying may be performed by any method which is usually used for this application, and examples thereof include spray drying, freeze drying, vacuum drying, shelf drying, belt drying, and drum drying. Among them, spray drying and freeze drying are preferable in view of easy handling of the powder. In the mixture of cartilage components containing proteoglycan obtained in the first embodiment, proteoglycan preferably has a molecular weight of 800 k to 900 k.

### Second Embodiment

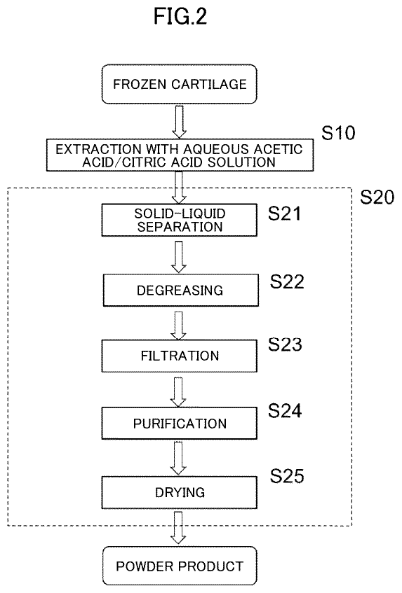

By a production method of the second embodiment of the present invention, cartilage components are extracted using a low-concentration aqueous acetic acid solution. FIG. 2 is a flowchart of a typical embodiment of this production method. The method of the second embodiment includes: an extraction step (S10) of immersing frozen cartilage in an aqueous acetic acid solution to obtain a cartilage component extract; and a collection step (S20) of collecting cartilage components from the extract.

The lower limit of the concentration of acetic acid in the aqueous acetic acid solution used in the extraction step (S10) is suitably 0.03% by mass or more, preferably 0.05% by mass or more, more preferably 0.1% by mass or more, yet more preferably 0.25% by mass or more, in view of higher extraction efficiency of proteoglycan. The upper limit of the concentration of acetic acid in the aqueous acetic acid solution is suitably less than 4% by mass, preferably 3.5% by mass or less, more preferably 3.1% by mass or less, yet more preferably 2% by mass or less, in view of higher extraction efficiency of collagen that is extracted simultaneously with proteoglycan.

More specifically, the collection step (S20) includes a solid-liquid separation step (S21) of removing the remaining cartilage, a degreasing step (S22) of removing lipids and other substances from the collected extract, a filtration step (S23), a purification step (S24), and a drying step (S25).

In the degreasing step (S22), components such as lipids which are considered to be mixed are simply removed from the proteoglycan extract by absorption using powdered cellulose and/or an oil absorbing mat. In the filtration step (S23), lipids and other substances are removed by an ordinary method using filter paper to obtain an extract from which the liquids are removed. A fine mesh or an ultrafiltration membrane may also be used. For example, the extract is collected by solid-liquid separation using a separation membrane having an appropriate molecular weight cutoff. After insoluble matters are removed by an ordinary method using a magnet trap, for example, the obtained filtrate may be solidified with a vacuum freeze dryer in the drying step (S25). Alternatively, the filtrate may be dried with a spray dryer to obtain a powdery solid.

In the mixture of cartilage components containing proteoglycan obtained in the second embodiment, proteoglycan preferably has a molecular weight of 400 k to 650 k.

### Third Embodiment

By a production method of the third embodiment of the present invention, cartilage components are extracted using a low-concentration aqueous citric acid solution in place of the aqueous acetic acid solution used in the second embodiment. In the same manner as the method of the second embodiment shown in FIG. 2, the method of the present embodiment also includes an immersion step (S10) of immersing frozen cartilage in a low-concentration aqueous citric acid solution to obtain a cartilage component extract and a collection step (S20) of collecting cartilage components from the extract.

The lower limit of the concentration of citric acid in the aqueous citric acid solution used in the extraction step (S10) is preferably 0.01% by mass or more, more preferably 0.015% by mass or more, yet more preferably 0.02% by mass or more, in view of higher extraction efficiency of proteoglycan. The upper limit of the concentration of citric acid in the aqueous citric acid solution is preferably less than 0.05% by mass, more preferably 0.049% by mass or less, yet more preferably 0.048% by mass or less, even more preferably 0.047% by mass or less, still more preferably 0.046% by mass or less, in view of higher extraction efficiency of collagen that is extracted simultaneously with proteoglycan.

The lower limit of the temperature of the aqueous citric acid solution during the extraction is preferably 30 degrees C. or more, more preferably 33 degrees C. or more, yet more preferably 35 degrees C. or more, in view of keeping the immersion liquid from decaying. The upper limit of the temperature of the aqueous citric acid solution during the immersion is preferably 80 degrees C. or lower, more preferably 70 degrees C. or lower, yet more preferably 60 degrees C. or lower. The lower limit of the extraction time is preferably 10 hours or more, more preferably 15 hours or more, yet more preferably 20 hours or more. The upper limit of the extraction time is preferably 96 hours or less, more preferably 72 hours or less, yet more preferably 50 hours or less. The collection step (S20) is performed in the same manner as in the second embodiment.

In the mixture of cartilage components containing proteoglycan obtained in the third embodiment, proteoglycan preferably has a molecular weight of 500 k to 900 k.

### OTHER EMBODIMENTS

In the extraction step (S10) of the second and third embodiments, water or an aqueous solution having pH 2 to 4 may be used in place of the aqueous acetic acid solution and the aqueous citric acid solution. The pH is in the following range in view of higher extraction efficiency of proteoglycan.
The lower limit of the pH is preferably 2 or more, more preferably 2.1 or more, yet more preferably 2.2 or more, even more preferably 2.3 or more, still more preferably 2.4 or more.
The upper limit of the pH is preferably 4 or less, more preferably 3.9 or less, yet more preferably 3.8 or less, even more preferably 3.7 or less, still more preferably 3.6 or less.

The present invention will be described in further detail below by way of Examples. However, the present invention is not limited to Examples. In the following Examples, the unit “%” of the numerical values indicating the amounts of various components added means “% by mass.”

### Examples

### [Example 1] Production of Mixture of Soluble Cartilage Components

Nasal cartilage extracted from the head of Chum salmon frozen and stored at −30 degrees C. to −20 degrees C. was freeze-dried at a product temperature of 35 degrees C. or lower (the temperature of the nasal cartilage was kept at 35 degrees C. or lower), and the obtained dried product was pulverized with a pulverizer to obtain a pulverized product having a particle size that passes a 14-mesh sieve. In 550 L of 99% ethanol, 55 kg of citric acid was dissolved, 55 kg of the obtained pulverized cartilage product was added, and the mixture was stirred at 50 degrees C. for an hour. Using a centrifuge, the ethanol solution was removed from the pulverized cartilage product washed by the stirring, and the residue was put into 550 L of 99% ethanol again and stirred at 50 degrees C. for 30 minutes. After the ethanol solution was removed with a centrifugal dehydrator, the residue was put into 550 L of 99% ethanol again and stirred and washed at 50 degrees C. for 30 minutes. The pulverized product washed with ethanol was collected by filtration using a centrifuge, dried under reduced pressure at a heating temperature of 35 degrees C. or lower, and the obtained dried product was pulverized using a pulverizer to obtain about 40 kg of a pulverized product.

About 800 kg of purified water was added to the obtained pulverized product having a particle size that passes a 60-mesh sieve, and the mixture was wet pulverized using a precision emulsifying disperser CLEARMIX (M Technique Co., Ltd.). The treatment solution thus emulsified and dispersed was heated to 80 degrees C. and warmed for 30 minutes for heat sterilization, and then an eluate was filtered with a stainless steel mesh (150 μm). The aqueous solution obtained by the filtration was freeze-dried with a freeze-dryer (FDU-2100, Tokyo Rikakikai Co., Ltd.) to obtain (about 35 kg of) a mixture of soluble cartilage components containing proteoglycan.

### (Quantification of Proteoglycan)

The dried product obtained by the production method was precisely weighed about 1 g, and a phosphate buffer (pH 6.8) was added to prepare exactly 10 mL of a sample solution. Each of the sample solutions was passed through a 0.45 μm membrane filter, and high performance liquid chromatography (HPLC) was performed under the following operating conditions to calculate the amount of proteoglycan from a calibration curve of a standard. The calibration curve was prepared by drying a proteoglycan standard (salmon nasal cartilage-derived, FUJIFILM Wako Pure Chemical Industries, Ltd., 162-22131) for three hours in a vacuum desiccator (silica gel) at room temperature, precisely weighing the dried sample, and dissolving the weighed sample in the same phosphate buffer used for the sample solutions to prepare a standard solution for the preparation of the calibration curve. A molecular weight at the peak top of a calibration curve prepared using Shodex STANDARD P-82 (manufactured by Showa Denko Co., Ltd.) was obtained as a molecular weight marker.

### Operating Conditions

Analyzer: HPLC analyzer
Detector: differential refractometer (RID-10A manufactured by Shimadzu Corporation)
Column: gel filtration column (TSKgel G5000PWXL manufactured by Tosoh Corporation)
Column temperature: 40 degrees C.
Sample injection volume: 50 μL
Mobile phase: phosphate buffer (pH 6.8)
Flow rate: 0.5 mL/min

### (Quantification of Collagens)

As one of features, collagens contain hydroxyproline and hydroxylysine that are not contained in general proteins. Hydroxyproline is said to account for about 10% of the total amino acids in collagen, and the amount of collagen can be estimated by quantifying hydroxyproline (“Functional properties of natural collagen,” Leather Science, vol. 56, No. 2, pp. 71-79, 2010). The content of collagens was calculated by measuring the amount of hydroxyproline in the dried sample obtained by the above production method.

### (Results)

The mixture of soluble cartilage components prepared in Example 1 contained proteoglycan having a molecular weight of about 840 k, and the content of the proteoglycan was about 41%. The content of collagens in the mixture was 35% to 38%.

### [Test Example 1] Odor Comparison Test of Mixture of Cartilage Components Containing Proteoglycan

Sensory tests were performed to compare odors of the products of Comparative Examples 1 to 4 and variations of Example 1 (Nos. 1 and 2) described below.

First, mixtures of cartilage components containing proteoglycan (pulverized products) of Comparative Examples 1 to 4 and the variations of Example 1 (Nos. 1 and 2) were prepared.

### [Comparative Example 1] Production of Pulverized Product

Nasal cartilage extracted from the head of Chum salmon frozen and stored at −30 degrees C. to −20 degrees C. was freeze-dried at a product temperature of 35 degrees C. or lower, and the obtained dried product was pulverized with a pulverizer to obtain a pulverized product having a particle size that passes a 14-mesh sieve. To 550 L of a 99% hexane solution, 55 kg of the obtained pulverized cartilage product was added, and the mixture was stirred at 25° C. (room temperature) for an hour. Using a centrifuge, the hexane solution was removed from the pulverized cartilage product washed by the stirring, and the residue was put into 550 L of a 99% hexane solution again and stirred at 25 degrees C. for 30 minutes. After the hexane solution was removed with a centrifuge, the residue was put into 550 L of a 99% hexane solution again and stirred and washed at 25 degrees C. for 30 minutes. The pulverized product washed with the hexane solution was collected by filtration using a centrifuge, dried under reduced pressure at a heating temperature of 35 degrees C. or lower, and the obtained dry product was pulverized using a pulverizer to obtain about 40 kg of a pulverized product.

### [Comparative Example 2] Production of Pulverized Product

Nasal cartilage extracted from the head of Chum salmon frozen and stored at −30 degrees C. to −20 degrees C. was freeze-dried at a product temperature of 35 degrees C. or lower, and the obtained dried product was pulverized with a pulverizer to obtain a pulverized product having a particle size that passes a 14-mesh sieve. To 550 L of a 99% acetone solution, 55 kg of the obtained pulverized cartilage product was added, and the mixture was stirred at 25 degrees C. for an hour. Using a centrifuge, the hexane solution was removed from the pulverized cartilage product washed by the stirring, and the residue was put into 550 L of a 99% acetone solution again and stirred at 25 degrees C. for 30 minutes. After the acetone solution was removed with a centrifuge, the residue was put into 550 L of a 99% acetone solution again and stirred and washed at 25 degrees C. for 30 minutes. The pulverized product washed with the acetone solution was collected by filtration using a centrifuge, dried under reduced pressure at a heating temperature of 35 degrees C. or lower, and the obtained dry product was pulverized using a pulverizer to obtain about 40 kg of a pulverized product.

### [Comparative Example 3] Production of Pulverized Product

Nasal cartilage extracted from the head of Chum salmon frozen and stored at −30 degrees C. to −20 degrees C. was freeze-dried at a product temperature of 35 degrees C. or lower, and the obtained dried product was pulverized with a pulverizer to obtain a pulverized product having a particle size that passes a 14-mesh sieve. To 550 L of a 59% ethanol solution, 55 kg of the obtained pulverized cartilage product was added, and the mixture was stirred at 25 degrees C. for an hour. Using a centrifuge, the ethanol solution was removed from the pulverized cartilage product washed by the stirring, and the residue was put into 550 L of a 59% solution again and stirred at 25° C. for 30 minutes. After the ethanol solution was removed with a centrifuge, the residue was put into 550 L of a 59% ethanol solution again and stirred and washed at 25° C. for 30 minutes. The pulverized product washed with the ethanol solution was collected by filtration using a centrifuge, dried under reduced pressure at a heating temperature of 35 degrees C. or lower, and the obtained dry product was pulverized using a pulverizer to obtain about 40 kg of a pulverized product.

### [Comparative Example 4] Production of Pulverized Product

Nasal cartilage extracted from the head of Chum salmon frozen and stored at −30 degrees C. to −20 degrees C. was freeze-dried at a product temperature of 35 degrees C. or lower, and the obtained dried product was pulverized with a pulverizer to obtain a pulverized product having a particle size that passes a 14-mesh sieve. To 550 L of a 99% ethanol solution, 55 kg of the obtained pulverized cartilage product was added, and the mixture was stirred at 25 degrees C. for an hour. Using a centrifuge, the ethanol solution was removed from the pulverized cartilage product washed by the stirring, and the residue was put into 550 L of a 99% solution again and stirred at 25 degrees C. for 30 minutes. After the ethanol solution was removed with a centrifuge, the residue was put into 550 L of a 99% ethanol solution again and stirred and washed at 25 degrees C. for 30 minutes. The pulverized product washed with the ethanol solution was collected by filtration using a centrifuge, dried under reduced pressure at a heating temperature of 35 degrees C. or lower, and the obtained dry product was pulverized using a pulverizer to obtain about 40 kg of a pulverized product.

### [First Variation of Example 1] Production of Pulverized Product

Nasal cartilage extracted from the head of Chum salmon frozen and stored at −30 degrees C. to −20 degrees C. was freeze-dried at a product temperature of 35 degrees C. or lower, and the obtained dried product was pulverized with a pulverizer to obtain a pulverized product having a particle size that passes a 14-mesh sieve. In 550 L of 99% ethanol, 55 kg of citric acid was dissolved, 55 kg of the obtained pulverized cartilage product was added, and the mixture was stirred at 60 degrees C. for 30 minutes. The stirring was performed simultaneously with wet-pulverizing using a homomixer. Using a centrifugal dehydrator, the ethanol solution was removed from the pulverized cartilage product washed by the stirring, and the residue was put into 550 L of 99% ethanol again and stirred at 60 degrees C. for 30 minutes. After the ethanol solution was removed with a centrifugal dehydrator, the residue was put into 550 L of 99% ethanol again and stirred and washed at 60 degrees C. for 30 minutes. The pulverized product washed with ethanol was collected by filtration using a centrifuge, dried under reduced pressure at a heating temperature of 35 degrees C. or lower, and the obtained dried product was pulverized using a pulverizer to obtain about 40 kg of a pulverized product.

### [Second Variation of Example 1] Production of Pulverized Product

Nasal cartilage extracted from the head of Chum salmon frozen and stored at −30 degrees C. to −20 degrees C. was freeze-dried at a product temperature of 35 degrees C. or lower, and the obtained dried product was pulverized with a pulverizer to obtain a pulverized product having a particle size that passes a 14-mesh sieve. To 550 L of 99% ethanol, 55 kg of the obtained pulverized cartilage product was added, and the mixture was stirred at 60 degrees C. for an hour. The stirring was performed simultaneously with wet-pulverizing using a precision emulsifying disperser CLEARMIX (M Technique Co., Ltd.). Using a centrifuge, the ethanol solution was removed from the pulverized cartilage product washed by the stirring, and the residue was put into 550 L of 99% ethanol again and stirred at 60 degrees C. for 30 minutes. After the ethanol solution was removed with a centrifuge, the residue was put into 550 L of 99% ethanol again and stirred and washed at 60 degrees C. for 30 minutes. The pulverized product washed with ethanol was collected by filtration using a centrifuge, dried under reduced pressure at a heating temperature of 35 degrees C. or lower, and the obtained dried product was pulverized using a pulverizer to obtain about 40 kg of a pulverized product.

Sensory tests of the pulverized products of Example 1, variations of Example 1, and Comparative Examples 1 to 4 were conducted (to check whether the products had odor emitted by lipids or any other substances). The sensory tests were performed with reference to, for example, the method described in “Nippon Shokuhin Kagaku Kogaku Kaishi Vol. 43, No. 12, pp. 1314 to 1322 (1996).” Table 1 shows the results of the sensory tests.

TABLE 1
Results of Sensory Test

| | Odor sensed |
|---|---|
| Groups tested | or not |
| Comparative Example 1 (washed with hexane) | ∘ |
| Comparative Example 2 (washed with acetone) | Δ |
| Comparative Example 3 (washed with 59% ethanol) | ∘ |
| Comparative Example 4 (washed with ethanol) | ∘ |
| First variation of Example 1 (washed by wet- | x |
| pulverizing with 5% citric acid ethanol) | |
| Second variation of Example 1 (washed by wet- | x |
| pulverizing with ethanol) | |

In Table 1, a circle symbol indicates “odor sensed,” a triangle symbol indicates “odor remained,” and a cross symbol indicates “no odor.” Table 1 suggests that the odor was removed from the products of the first and second variations of the first embodiment.

### [Examples 2 to 9] Production of Powder Mixture of Proteoglycan and Collagens by Extraction Using Low-Concentration Acetic Acid

As a starting material, 400 g of nasal cartilage extracted from the head of Chum salmon frozen and stored at −30 degrees C. to −20 degrees C. was prepared. To the starting material, aqueous acetic acid solutions having different concentrations, 2,000 mL each, were added, followed by extraction at an extraction temperature of 30 degrees C. to 40 degrees C. for 48 hours to 72 hours. Table 2 show the concentrations (%), extraction temperatures (° C.), and extraction time (hr., hours).

Each of the obtained extracts were filtered through a filter paper No. 26 (110 mm) to remove insoluble matters. Then, powdered cellulose (trade name: “KC Flock W-400G,” manufactured by Nippon Paper Industries Co., Ltd.) was added at 2% of the volume of the extract, and the mixture was stirred for 30 minutes, followed by filtration. The filtrate was concentrated using a hollow fiber membrane with a molecular weight cutoff of 50 k until the volume of the filtrate was reduced to 1/10. Concentration and purification were repeated while diluting the product with water to finally obtain 500 g to 800 g of a concentrate (pH 6 to 7). Then, the obtained concentrate was freeze-dried to obtain 10 g to 20 g of a mixture (freeze-dried product) of proteoglycan and collagens.

Using the method described in Example 1, the following items were measured for the obtained freeze-dried products. Table 2 shows the results.
Yield of PG (%): a value (%) calculated by [(content of proteoglycan in the obtained freeze-dried product)/400 (g, weight of the nasal cartilage)]×100
Molecular weight of PG (×10⁴): molecular weight of proteoglycan For example, the entry “56” in the column of Example 2 in Table 2 indicates a molecular weight of 560 k (560,000).
Content ratio of PG: a relative value of the proteoglycan content in the freeze-dried product of each Example to the proteoglycan content in the freeze-dried product of Comparative Example 5 regarded as 100 (a relative value compared with Comparative Example 5)
Content of collagen (%): content of collagens in the freeze-dried product
“-”: Not measured

TABLE 2

| | Comp. | | | | | | | | |
|---|---|---|---|---|---|---|---|---|---|
| | Ex. 5 | Ex. 2 | Ex. 3 | Ex. 4 | Ex. 5 | Ex. 6 | Ex. 7 | Ex. 8 | Ex. 9 |
| Concentration of acetic acid (%) | 0.025 | 0.5 | 0.5 | 1.0 | 1.0 | 2.0 | 2.0 | 2.0 | 3.0 |
| Extraction temperature (° C.) | 37 | 37 | 37 | 37 | 37 | 37 | 37 | 37 | 37 |
| Extraction time (hr.) | 48 | 48 | 72 | 48 | 72 | 48 | 72 | 48 | 48 |
| Yield of solid content (%) | 0.16 | 4.87 | 4.61 | 4.78 | 5.16 | 3.32 | 3.96 | 3.75 | 2.99 |
| Yield of PG (%) | 0.05 | 3.84 | 3.4 | 2.75 | 3.63 | 2.54 | 2.72 | 3.33 | 2.58 |
| Molecular weight of PG (×10⁴) | 55 | 56 | 52 | 60 | 57 | 60 | 55 | 50 | 52 |
| Content ratio of PG | 100 | 246.11 | 229.28 | 177.57 | 219.00 | 237.69 | 237.69 | 276.64 | 225.55 |
| (Content relative to Comparative | | | | | | | | | |
| Example 5) | | | | | | | | | |
| Content of collagen (%) | — | — | 26.33 | — | 33.01 | — | — | 26.02 | — |

It was confirmed that the products of Examples 2 to 9 improved the yield of proteoglycan as compared with the product of Comparative Example 5.

### [Examples 10 to 12] Production of Proteoglycan by Extraction Using Low-Concentration Citric Acid

As a starting material, 400 g of nasal cartilage extracted from the head of Chum salmon frozen and stored at −30 degrees C. to −20 degrees C. was prepared. To the starting material, aqueous acetic acid solutions having different concentrations, 2,000 mL each, were added, followed by extraction at different extraction temperatures for 24 hours to 48 hours while the mixture was slowly stirred. Table 3 shows the concentrations (%), extraction temperatures (° C.), and extraction time (hr., hours).

Each of the obtained extracts was filtered through a filter paper No. 65 (110 mm) to remove insoluble matters. Then, powdered cellulose (trade name: “KC Flock W-400G,” manufactured by Nippon Paper Industries Co., Ltd.) was added at 2% of the volume of the extract, and the mixture was stirred for 30 minutes, followed by filtration. The filtrate was concentrated using a hollow fiber membrane with a molecular weight cutoff of 50 k until the volume of the filtrate was reduced to 1/10. Concentration and purification were repeated while diluting the product with water to finally obtain 500 g to 800 g of a concentrate (pH 6 to 7). Then, the obtained concentrate was freeze-dried to obtain 10 g to 20 g of a mixture of proteoglycan and collagens.

Using the method described in Example 1, the following items were measured for the obtained freeze-dried products. Table 3 shows the results.
Yield of PG (%): a value (%) calculated by [(content of proteoglycan in the obtained freeze-dried product)/400 (g, weight of the nasal cartilage)]×100
Molecular weight of PG (×10⁴): molecular weight of proteoglycan For example, the entry “72” in the column of Example 10 in Table 2 indicates a molecular weight of 720 k (720,000).
Ratio of PG/collagen=(content of proteoglycan in the freeze-dried product/content of collagen in the freeze-dried product)
Content of collagen (%): content of collagens in the freeze-dried product
“-”: Not measured
Whether the product decayed or not: a cross symbol indicates a group of the freeze-dried products that decayed during the production, and a circle symbol indicates a group of the freeze-dried products that had no decay during the production (a normal group). The product of Comparative Example 6 decayed, and thus the PG yield and the collagen content could not be measured.

TABLE 3

| | Comp. | | | |
|---|---|---|---|---|
| | Ex. 6 | Ex. 10 | Ex. 11 | Ex. 12 |
| Concentration of citric acid (%) | 0.050 | 0.025 | 0.025 | 0.045 |
| Extraction temperature (° C.) | 22 | 50 | 50 | 37 |
| Extraction time (hr.) | 48 | 24 | 48 | 24 |
| Decayed or not | x | ∘ | ∘ | ∘ |
| Yield of PG (%) | — | 2.44 | 2.78 | 2.73 |
| Molecular weight of PG (×10⁴) | — | 72 | 81 | 62 |
| PG/collagen ratio | — | 8.80 | 8.90 | — |
| Content of collagen (%) | — | 11.37 | 11.24 | — |

### [Test Example 2] Human Fibroblast Proliferation Test (No. 1)

The mixture of soluble cartilage components obtained in Example 1 was evaluated for the ability of human fibroblast proliferation.

First, DMEM (D-MEM (low glucose) with L-glutamine and phenol red, 041-29775, FUJIFILM Wako Pure Chemical Industries, Ltd.) was prepared. Into a 96-well plate, 4×10³ cells suspended in 200 μl DMEM containing 0.1% FBS were seeded. After the seeding, the cells were cultured in an environment of 37 degrees C. and 5% CO₂ for 72 hours. After the culturing, the medium was replaced with 200 μl DMEM containing 0.1% FBS. After the replacement, the cells were cultured in an environment of 37 degrees C. and 5% CO₂ for 24 hours. After the culturing for 24 hours, the medium was replaced with a medium containing the sample (200 μl DMEM containing 0.1% FBS). At that time, a control group in which the cells were cultured only in DMEM containing 0.1% FBS with no predetermined sample added was prepared. Then, the cells were cultured in an environment of 37 degrees C. and 5% CO₂ for 72 hours. After the culturing, the cell proliferation effect of the added samples (groups of Samples 1 to 3) was evaluated using CellTiter-Glo (trademark) Luminescent Cell Viability Assay (Promega). For the test results obtained, statistical significance was assessed using Dunnett's test. In Table 4, a symbol “**” was added when a significant difference was observed (p<0.05). The cell proliferation effect was evaluated by measuring the number of normal human dermal fibroblasts of each group. For the measurement, a relative value of the number of cells measured in each group (groups of samples 1 to 3) was calculated regarding the measured number of cells in the control group as 100. Table 4 below shows the relative values using the results obtained by calculating the average values of three samples of each group.
Control group: a group cultured in DMEM containing 0.1% FBS with no sample added
Group of sample 1: five-times concentrated proteoglycan contained in proteoglycan F (manufactured by ICHIMARU PHARCOS Co., Ltd.) as a conventional product
Group of sample 2: mixture of soluble cartilage components prepared in Example 1
Group of sample 3: mixture of soluble cartilage components prepared in Example 5
Group of samples 4: commercially available proteoglycan

TABLE 4

| Concentration of | | | | |
|---|---|---|---|---|
| sample added | Relative value of fibroblast number | | | |
| (ug/mL) | Sample 1 | Sample 2 | Sample 3 | Sample 4 |
| 50 | 124.8 (**) | 143.7 (**) | 117.6 (**) | 114.8 |
| 100 | 140.2 (**) | 142.2 (**) | 119.9 (**) | 119.9 (**) |

The groups of samples 1 to 4 showed the higher fibroblast proliferation ability than the control group. In particular, the groups of samples 1 to 3 significantly showed the fibroblast proliferation ability (p<0.05, as indicated by the symbol ** in Table 4) at any addition concentration.

### [Test Example 2] Human Fibroblast Proliferation Test

Proteoglycans obtained in Examples 10 and 11 were evaluated for the ability of human fibroblast proliferation in the same manner as in Test Example 1. The following samples were used. For the test results obtained, statistical significance was assessed using Dunnett's test. Table 5 shows the measurement results. In Table 5, a symbol “**” was added when a significant difference was observed (p<0.05). The cell proliferation effect was evaluated by measuring the number of normal human dermal fibroblasts of each group. For the measurement, a relative value of the number of cells measured in each group (groups of samples 1, 5, and 6) was calculated regarding the measured number of cells in the control group as 100. Table 5 below shows the relative values using the results obtained by calculating the average values of three samples of each group.
Group of sample 1: five-times concentrated proteoglycan contained in proteoglycan F (manufactured by ICHIMARU PHARCOS Co., Ltd.) as a conventional product
Group of sample 5: Proteoglycan prepared in Example 10
Group of samples 6: Proteoglycan prepared in Example 11

TABLE 5

| Concentration of | | | |
|---|---|---|---|
| sample added | Relative value of fibroblast number | | |
| (ug/mL) | Sample 1 | Sample 5 | Sample 6 |
| 50 | 127 (**) | 114 | 114 |
| 100 | 138 (**) | 119 | 117 |

All the samples (samples 1, 5, and 6) showed the higher fibroblast proliferation ability than the control group. A significant difference (p<0.05) was found in the group of sample 1.

### [Examples 13 and 14] Production of Powder Mixture of Proteoglycan and Collagens

As a starting material, 400 g of nasal cartilage extracted from the head of Chum salmon frozen and stored at −30 degrees C. to −20 degrees C. was prepared. To the starting material, water or aqueous solutions with pH 2 to 4, 2,000 mL each, were added, followed by extraction at an extraction temperature of 37 degrees C. for 72 hours. Table 6 shows the concentrations (%), extraction temperatures (° C.), and extraction time (hr., hours).

Each of the obtained extracts were filtered through a filter paper No. 26 (110 mm) to remove insoluble matters. Then, powdered cellulose (trade name: “KC Flock W-400G,” manufactured by Nippon Paper Industries Co., Ltd.) was added at 2% of the volume of the extract, and the mixture was stirred for 30 minutes, followed by filtration. The filtrate was concentrated using a hollow fiber membrane with a molecular weight cutoff of 50 k until the volume of the filtrate was reduced to 1/10. Concentration and purification were repeated while diluting the product with water, and pH control was performed (using a solution shown in Table 6) to finally obtain 500 g to 800 g of a concentrate. Then, the obtained concentrate was freeze-dried to obtain 10 g to 20 g of a mixture (freeze-dried product) of proteoglycan and collagens.

Using the method described in Example 1, the following items were measured for the obtained freeze-dried products. Table 6 below shows the results.
Yield of PG (%): a value (%) calculated by [(content of proteoglycan in the obtained freeze-dried product)/400 (g, weight of the nasal cartilage)]×100
Molecular weight of PG (×10⁴): molecular weight of proteoglycan For example, the entry “50” in the column of Example 13 in Table 6 indicates a molecular weight of 500 k (500,000).
Content of collagen (%): content of collagens in the freeze-dried product
“-”: Not measured

TABLE 6

| | Example 13 | Example 14 |
|---|---|---|
| pH of water | 2.68 | 2.68 |
| Extraction temperature (° C.) | 37 | 37 |
| Extraction time (hr.) | 72 | 72 |
| Yield of PG (%) | 3.33 | 3.1 |
| Molecular weight of PG (×10⁴) | 50 | 50 |
| Solution used for pH control | NaOH solution | KOH solution |
| Content of collagen (%) | — | 25.24 |

In the same manner as in Example 4, the mixtures (freeze-dried products) of Examples 13 and 14 were obtained with certain proteoglycan yields.

[Others] pH of Solvents used in Examples and Comparative Examples shown in

### Tables 2 and 3

The pHs of the solvents used in Comparative Example 5 and Examples 2 to 9 (Table 2, 2,000 mL of aqueous acetic acid solutions of predetermined concentrations) and the solvents used in Comparative Example 6 and Examples 10 to 12 (Table 3, 2,000 mL of aqueous citric acid solutions of predetermined concentrations) were measured. Table 7 below shows the measurement results.

TABLE 7

| | | pH of |
|---|---|---|
| | Solvent | solvent |
| Comparative | 0.025% aqueous acetic acid solution | 3.63 |
| Example 5 | | |
| Example 2 | 0.5% aqueous acetic acid solution | 2.83 |
| Example 3 | 0.5% aqueous acetic acid solution | 2.83 |
| Example 4 | 1.0% aqueous acetic acid solution | 2.68 |
| Example 5 | 1.0% aqueous acetic acid solution | 2.68 |
| Example 6 | 2.0% aqueous acetic acid solution | 2.51 |
| Example 7 | 2.0% aqueous acetic acid solution | 2.51 |
| Example 8 | 2.0% aqueous acetic acid solution | 2.51 |
| Example 9 | 3.0% aqueous acetic acid solution | 2.41 |
| Comparative | 0.05% aqueous citric acid solution | 2.87 |
| Example 6 | | |
| Example 10 | 0.025% aqueous citric acid solution | 2.89 |
| Example 11 | 0.025% aqueous citric acid solution | 2.89 |
| Example 12 | 0.045% aqueous citric acid solution | 3.16 |

### INDUSTRIAL APPLICABILITY

The production method of the present invention can efficiently extract and collect cartilage components including proteoglycan from cartilage. A mixture of cartilage components produced by this method also contains other components than proteoglycan such as collagen and hyaluronic acids, and can be used as a raw material for foods and cosmetics.

1. A method for producing a mixture of cartilage components containing proteoglycan, the method comprising:
pulverizing freeze-dried cartilage to obtain a pulverized product;
washing the pulverized product with an ethanol solution containing citric acid;
washing the product washed with the ethanol solution containing the citric acid further with ethanol;
drying and pulverizing the product washed with the ethanol to obtain powder; and
wet-pulverizing the obtained powder in water.

2. The method of claim 1, wherein
the ethanol solution containing the citric acid contains the citric acid from greater than or equal to 1% by mass to less than or equal to 30% by mass.

3. The method of claim 1, wherein
the mixture of the cartilage components contains the proteoglycan of 30% by mass or more on a solids basis.

4. The method of claim 1, wherein
the mixture of the cartilage components contains water-soluble collagens of at least 20% by mass on a solids basis.

5. The method of claim 1, wherein
a molecular weight of the proteoglycan is 800 k to 900 k.

6. A method for producing a mixture of cartilage components containing proteoglycan, the method comprising:
immersing cartilage in an aqueous acetic acid solution with a concentration of acetic acid in water of 0.03% or more to less than 4% by mass to obtain a cartilage component extract; and
collecting the cartilage components from the extract.

7. The method of claim 6, wherein
the concentration of the acetic acid in water of the aqueous acetic acid solution is 0.5% to 3% by mass.

8. The method of claim 6, wherein
the mixture of the cartilage components contains collagens, a content of the collagens being at least 20% by mass on a solids basis.

9. The method of claim 6, wherein
a molecular weight of the proteoglycan is 400 k to 650 k.

10. A method for producing a mixture of cartilage components containing proteoglycan, the method comprising:
immersing cartilage in an aqueous citric acid solution with a concentration of citric acid in water of 0.01% or more to less than 0.05% by mass at 30 degrees C. to 80 degrees C. to obtain a cartilage component extract; and
collecting the cartilage components from the extract.

11. The method of claim 10, wherein
a molecular weight of the proteoglycan is 500 k to 900 k.

12. The method of claim 1, wherein
the cartilage is nasal cartilage of a salmon head.

13. A method for producing a mixture of cartilage components containing proteoglycan, the method comprising:
immersing cartilage in water or an aqueous solution of pH 2 to 4 to obtain a cartilage component extract; and
collecting the cartilage components from the extract.

14. The method of claim 6, wherein the cartilage is nasal cartilage of a salmon head.

15. The method of claim 10, wherein the cartilage is nasal cartilage of a salmon head.
